(19) United States
(12) Patent Application Publication
(10) Pub. No.: US 2005/0192592 A1
(43) Pub. Date: Sep. 1, 2005

(54) Self-tensioning handle for endoscopic device

(75) Inventors: Gary L. Butler, Bloomington, IN (US); Thomas L. Foster, Poland, IN (US); Frank J. Fischer JR., Bloomington, IN (US); Nicholas G. Terwiske, Elletsville, IN (US); John H. Ward, Bloomington, IN (US); Richard E. Stamper, Terre Haute, IN (US); David Radue, Terre Haute, IN (US); Elizabeth Purdy, Terre Haute, IN (US); Robert Davignon, Terre Haute, IN (US); Barry Davignon, Terre Haute, IN (US); Ronald Hofmann, Brazil, IN (US)

Correspondence Address: BRINKS HOFER GILSON & LIONE, P.O. BOX 10395, CHICAGO, IL, 60610, US

(73) Assignees: Cook Urological Incorporated; Stamper Medical Technologies, Inc.

(21) Appl. No.: 11/065,220

(22) Filed: Feb. 24, 2005

Related U.S. Application Data
(60) Provisional application No. 60/548,674, filed on Feb. 27, 2004.

(51) Int. Cl. A61B 17/24

(52) U.S. Cl. 606/114

(57) ABSTRACT

A self-tensioning handle for use with endoscopic devices is disclosed. The handle may have mounting features for mounting a sheath and a control rod from an endoscopic device, such as a grasper or a retrieval basket. One mounting feature, such as a pin vise to hold a sheath, may be mounted in a distal portion of the handle, nearer a patient. Another mounting feature for a control rod or cannula of the endoscopic device is preferably mounted in a proximal portion of the handle. The handle is assembled with a tension or a pre-load so that a surgeon is not required to continually grip and compress the handle to retain a sample in the retrieval basket or other end-effector used with the handle.

This application claims the benefit of the filing date under 35 U.S.C. § 119(e) of Provisional U.S. Patent Application Ser. No. 60/548,674, filed on Feb. 27, 2004, which is hereby incorporated by reference in its entirety.

### FIELD OF THE INVENTION

This invention relates generally to surgical devices and more particularly to a handle for surgical devices. The handle relates to devices for capturing and retrieving or extracting stones, calculi, concretions, foreign bodies and the like from a human or veterinary patient. The handle may also be used for many other medical purposes, such as retracting or manipulating body tissues.

### BACKGROUND OF THE INVENTION

Various organs and passages in the body are subject to the development of stones, calculi and the like. For example, kidney stones are a common problem in the United States. Kidney stones are painful and are the most frequent cause of kidney inflammation. Calculi and concretions in other parts of the biliary system are also commonplace. Similarly, stones, calculi, concretions and the like can develop throughout the renal or urinary system, not only in the ureters and distal to them, but also in the renal tubules and in the major and minor renal calyxes.

Minimally invasive surgical procedures have been developed for the removal of stones, calculi, concretions and the like from the biliary, vascular, and urinary systems, as well as for the removal or retrieval of foreign bodies from a variety of locations in the body. Such procedures avoid the performance of open surgical procedures such as, for example, an anatrophic nephrolithotomy. Minimally invasive procedures can instead employ percutaneous access, in which stones, calculi, concretions, foreign bodies and the like are removed through a percutaneously inserted access sheath. Several access routes are suitable, depending upon the specific system and the particular location in the system at which the stones, calculi, concretions, foreign bodies or the like are found. One access route that is infrequently used is direct percutaneous insertion of a retrieval device to remove calculi and kidney stones.

Without regard to the particular access route, percutaneous extraction may be based upon the use of catheters or similar devices to engage and remove the stones, calculi, concretions, foreign bodies and the like. Such catheters and devices typically comprise a hollow, flexible sheath and an end effector at the distal end of an inner cannula. The end-effector may be a basket comprising a plurality of wires positioned in and extendable from the sheath. The wires are joined or arranged so as to form a basket or forceps for engaging the object to be retrieved when the wires are extended from the sheath. The basket can be collapsed by withdrawing the wires into the sheath. A helical basket permits entry of the stone or the like from the side of the basket, while an open ended (“eggwhip”) basket allows a head-on approach to the stone or the like. Other retrievers and graspers can include forceps or can include a loop or snare for encircling the body to be removed, the loop or snare being made of the wire. Such devices may be used in conjunction with a nephroscope, to aid the physician in seeing the operating field. Using such a device also tends to limit the size of the cannula and basket used.

Despite their successful use for some time, such retrieval devices are subject to drawbacks. The principal device that is used to retrieve kidney stones is a 3-pronged grasper. The prongs of the grasper, useful in grasping stones, may cause damage to kidney or contiguous tissue, leading to bleeding, and potentially significantly extending the time for the procedure. The very flexible, movable nature of these graspers adds to the problem, in that their flexibility and mobility make them more difficult to control. One particular aspect that makes these devices difficult to control is the need for the surgeon to constantly grip or flex the handle in order to control the basket or other end effector during operation. The need for constant flexing is tiring during a long procedure and contributes to surgeon fatigue.

It would be highly desirable to have a device that is easier to control when used inside the human body for the capture and retrieval or extraction of kidney stones, or for a variety of other medical procedures. The device would ideally also be suitable for manipulating tissue or other objects inside the body.

### BRIEF SUMMARY OF THE INVENTION

The foregoing problems are solved and a technical advance is achieved in a self-tensioning handle useful for capturing and extracting, retrieving or removing objects such as stones and calculi from the human body, and from kidneys in particular. Of course, the device is not limited to human bodies, but may also be used in veterinary applications. One embodiment is a self-tensioning handle for an endoscopic device, the handle comprising an upper portion, an intermediate portion mounted to the upper portion, and an actuating portion. The actuating portion is operably connected to the adjustable portion for operating the endoscopic device, wherein the handle is configured to fit in a palm of a user's hand and the upper, intermediate, and actuating portions are molded together.

Another embodiment is a self-tensioning actuator for an endoscopic device, the actuator comprising, means for fixedly mounting a first portion of the endoscopic device, means for slidably mounting a second portion of the endoscopic device, and means for adjustably actuating the endoscopic device. The means for fixedly mounting, the means for slidably mounting and the means for adjustably actuating include a top portion and are molded as a unit, wherein the actuator is configured to fit in a palm of a user's hand.

Another embodiment is a self-tensioning handle for an endoscopic device. The handle comprises an upper portion, an intermediate portion mounted to the upper portion, an adjustable portion mounted to the intermediate portion, and an actuating portion operably connected to the adjustable portion for operating the endoscopic device. The handle is configured to fit in a palm of a user's hand.

Another embodiment is a self-tensioning handle for controlling an endoscopic device. The handle comprises a proximal portion for mounting a first portion of the endoscopic device, a distal portion for mounting a second portion of the endoscopic device, and a squeezable actuation portion comprising at least two webs attached to the proximal and distal portions.

Another embodiment is a self-tensioning handle for an endoscopic device. The handle comprises a proximal portion for mounting a first portion of the endoscopic device. The handle also comprises a distal portion for mounting a second portion of the endoscopic device, and a bendable portion connecting the proximal and distal portions, and at least one control device for controlling movement of the endoscopic device. In this embodiment, the proximal, flexible and distal portions are molded together. Flexible plastics are preferred, such as polypropylene and polyethylene.

There are many ways to practice the present invention, a few of which are shown in the following drawings and specification. The embodiments described below are not meant to limit the invention, but rather to describe and illustrate the many ways that the present invention may be used. The advantages of the invention include better control over the endoscopic device used, as well as better devices themselves, leading to easier entry, less damage and bleeding, and shorter removal procedures.

### BRIEF DESCRIPTION OF THE FIGURES

The present invention will now be described in conjunction with the following drawings, wherein like reference characters refer to like parts throughout the several views.

FIG. 1 is a perspective view of a first embodiment of a self-tensioning handle with an endoscopic basket retriever or other instrument.

FIG. 2 is a perspective view of a second embodiment of a self-tensioning handle in combination with an endoscopic grasper-type end-effector.

FIGS. 3a through 3f are perspective views of different embodiments of slides that may be used with the embodiments of the handles shown in FIGS. 1 and 2.

FIG. 4 is a third embodiment of a self-tensioning handle for use with basket retrievers or other end-effectors.

FIG. 5 is a fourth embodiment of a self-tensioning handle, similar to the embodiment of FIG. 3.

FIG. 6 is a fifth embodiment of a self-tensioning handle.

FIG. 7 is a more detailed view of the embodiment of FIG. 6.

FIG. 8 is a sixth embodiment of a self-tensioning handle for use with endoscopic instruments.

FIG. 9 is a seventh embodiment of a self-tensioning handle for endoscopic instruments.

FIG. 10 is an eighth embodiment of a self-tensioning handle.

### DETAILED DESCRIPTION OF THE PRESENTLY PREFERRED EMBODIMENTS

The self-tensioning handle is useful because it requires attention and effort from the surgeon only periodically. That is, the surgeon need only flex his or her hand when actively using the end-effector at the distal end of the instrument. For instance, the end-effector may be a basket, and the surgeon may be using the basket to retrieve a kidney stone or a portion of a kidney stone. The surgeon need only squeeze the self-tensioning handle to extend the basket, or to retract the sheath, and then capture the stone. Once the stone is captured, typically viewed by an endoscope, the basket may be retracted, or the sheath extended, by the surgeon relaxing his or her hand. The self-tensioning aspect of the handle insures that the retraction of the basket, or the extension of the sheath, is virtually automatic. Thereafter, the stone is captured in the basket and the surgeon need only extract the entire instrument, basket and sheath. No further effort is required to retain the stone in the basket, because the handle does all the work once the capture is completed.

A first embodiment of an endoscopic device with a self-tensioning handle is depicted in FIG. 1. The handle 10 is depicted with an outer sheath 14, a control rod 15, and a retrieval basket 18. The wires 17 of retrieval basket 18 are secured to control rod 15 with a joint 16. Joint 16 may be a crimp as shown, or may be solder joint or braze joint, or any other suitable joint, such as a weld or even a medically-acceptable adhesive.

Self-tensioning handle 10 includes a top portion 10a, a front portion 10b with a mount 12 suitable for mounting outer sheath 14. Mount 12 may be a pin-vise or other mount suitable for fixedly mounting the outer sheath. Handle 10 also includes gripping portions 10c and 10d, which may be somewhat more flexible than other portions of the handle. Handle rear portions 10e and 10g are designed to fit in the palm of a user's hand. Middle portion 10f may be a bridge between top portion 10a and front portion 10d, with end portion 10h.

The handle also includes slide member 11 which may slide back and forth on intermediate portion 10f. Slide member 11 is connected to end portion 10h, as shown in the drawings. The handle works as follows. When molded and assembled, outer sheath 14 is held fixedly in place by mount 12. Control rod 15 is mounted to slide member 11, which is also mounted to end portion cylindrical portion interface 10h. The handle is molded such that the back portions, 10e, 10g, and 10h, exert flexure away from front portions 10b and 10d. The amount of flexure will vary with the thickness and width of the parts, and also with the material used for the handle. Because end portion 10h is connected to slide member 11, slide member 11 will be placed in tension, pulling slide member 11 rearward.

The tension may be overcome by a user gripping the handle and applying a force with his or her hand. Squeezing the handle will push portions 10c and 10e upward, pushing rear portion 10g upward. This will cause slide member 11 to translate to the right, or distally, in the direction of arrow A. An inner sheath 19 may also be attached to slide member 11, inner sheath 19 having a diameter somewhat greater than control rod 15. The inner sheath will extend sufficiently to prevent buckling of the control rod, but will not extend so far as to interfere with flexing or using the handle. Control rod 15, connected to the distal or right end of slide member 11, also translates to the right, the control rod leaving sheath 14 and deploying basket 18. In the embodiment of FIG. 1, the length of slide member 11 determines the “throw” or translation of the control rod and thus the end-effector or basket with respect to the outer sheath. Front portion 10b may also be considered to be a distal portion, and rear portion 10g may be considered to be a proximal portion. The rear of portion 10b may have a radiused section 10i to fit end portion 10h.

With this embodiment, a surgeon will position the distal end of the sheath near a kidney stone or basket, and then deploy the basket by squeezing the handle. The basket deploys, capturing the stone or stone fragments as the basket leaves the sheath. Once the fragment or fragments are captured, the surgeon is ready to extract them from the patient's body. The surgeon gently releases the handle, causing the sliding member to translate in a proximal direction, opposite to arrow A in FIG. 1, also causing the control rod to translate distally, and drawing the basket at least partially back into the sheath. Thus, the surgeon only squeezes the handle during the period of time required to capture material within the body. Afterwards, the surgeon may relax and concentrate on gently removing the device and the material from the patient's body.

The handle, the outer sheath, and the inner control rod cooperate to extend and retract the basket. The basket preferably is made so that it extends about 2.7 cm plus or minus 2 mm (about 1.05 inches plus or minus about 0.08 inches). Other extension ranges may be used. The basket will extend to the extent that the control rod is translated by the surgeon applying force to the handle and extending the control rod. Because the wires necessarily are not straight, but curve to form a basket, a somewhat greater translation of about 4 cm (about 1.6 inches) of the control rod may be necessary to extend a basket of about 2.7 cm (about 1.1 inches). In a preferred embodiment, when the basket extends about 2.7 cm (about 1.1 inches) from the end of the outer sheath, the width of the basket (diameter) is about 1.8 cm, plus or minus about 2 mm (about 0.71 inches plus or minus about 0.08 inches). Other configurations may be used.

The control rod is desirably made from a medically acceptable metal, such as stainless steel, so that the user has a high degree control over the maneuverability and extension of the control rod and the end-effector at the distal end of the control rod. As noted above, the control rod may be joined to the slide member at an interface, such as a braze joint or a crimp. The control rod may also be secured to the slide member with an adhesive, such as a medically-acceptable grade of cyanoacrylate adhesive. Loctite 4011 works well and is preferred.

The outer sheath is desirably made from a flexible, medically acceptable material such as polyimide or a fluorocarbon material, or other medically acceptable materials. The outer sheath may also be made from flexible metals, such as a coil spring, or thin metallic tubing that has been made flexible, such as by helical cuts along the length of the tubing, as disclosed in pending application Ser. No. 10/617,580, now U.S. Pat. No. ______, and which is hereby incorporated by reference. The outer sheath may be covered with a thin adherent plastic coating, in order to aid the surgeon in maneuvering the endoscopic instrument. The coating is desirably a medical grade plastic material, such as Teflon® (PTFE) or other grade of plastic or fluoropolymer. These may include FEP, fluorinated ethylene propylene, PFA, perfluoroalkoxy polymer, and other medically-acceptable grades of thermoplastic or thermoset coatings.

The wires used to form the basket may be stainless steel, or are preferably a superelastic shape-memory material, such as Nitinol, a Ni—Ti alloy. Other alloys, such as Cu—Zn—Al, or Cu—Al—Ni may also be used. Round wires are preferably used to form the basket, but triangular and flat wires may also be used. Wires having a diameter of from about 0.08 mm to about 0.15 mm (about 0.003 inches to about 0.006 inches) are preferred, because their use permits a very small diameter basket, and hence a small diameter cannula. It is also preferred that the wires and the small loops used to restrict movement of the wires be kink-free. This is achieved by using the shape-memory metals mentioned above, and heat treating them in the desired shape for a short period of time.

Shape-memory or superelastic materials are heat treated or annealed from a weak (martinsite) structure to a strong (austenite) structure. The alloys are weak and deformable in the martinsitic state, which is thus useful for forming the basket and the loops. After transformation to the strong or martensitic state, they exhibit a superelastic property so long as the material remains above a transformation temperature, at which temperature it will revert to the martensitic state. The transformation temperature is desirably a low temperature, well below the temperature of a human body, and preferably below room temperature, about 20-25° C. The transformation temperature of the wires and the basket is thus selected to be below the operating temperature of the basket, thus keeping the basket in a superelastic state. In this state, the wires advantageously return to their original, unstressed shape when deforming stresses are removed. The superelastic wire alloy also increasingly resists deformation as the stress load is increased. Thus, when a superelastic basket is collapsed and placed into the cannula, a stress load is placed on the basket. When the basket is deployed the stresses are removed, the basket returns to the desired shape, and may be used to encircle a stone or other desired object.

It was noted above that the dimensions of the handle (as in FIG. 1) determines the force that the surgeon uses to extend the basket from the sheath. If the handle, the control rod, the outer sheath, and the basket are relatively free of friction, then the potential energy stored in the “squeezed” handle is available for grasping a stone or other calculus. This force used to squeeze the handle is stored as potential energy in the deformation of the handle, much as energy is stored in a compressed spring. That energy or force is applied to the stone or calculus when the surgeon releases the handle and the potential energy is used to trap or “squeeze” the stone or calculus, or to operate another retrieval assembly at the distal end of the control rod. The force desired is typically that force which is sufficient to trap and hold, but not sufficient to crush or cut, the stone or calculus.

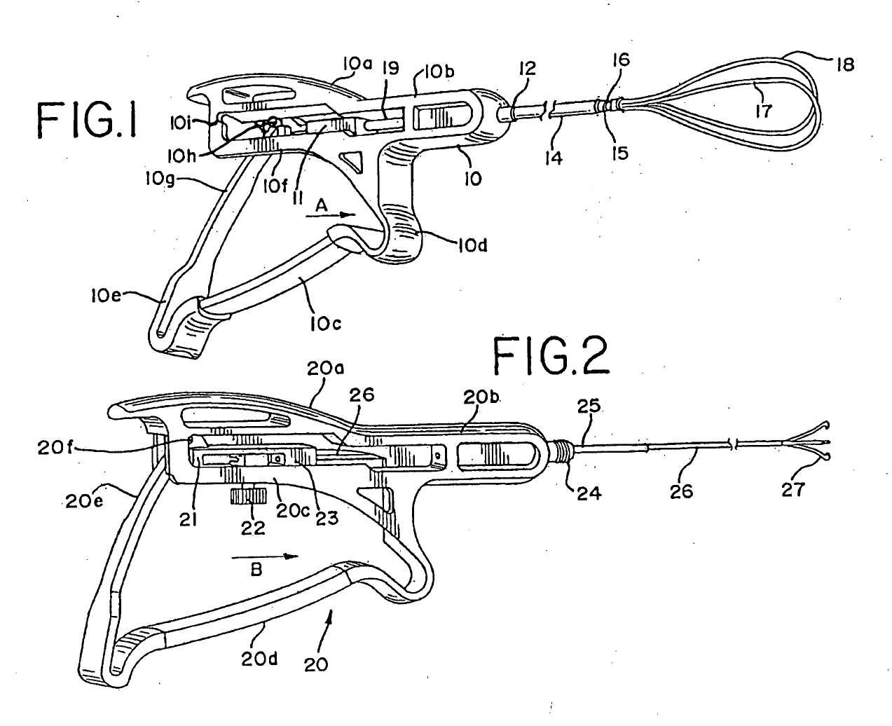

Another embodiment of a squeezable handle with built-in tension is depicted in FIG. 2. The embodiment of FIG. 2 is similar to that of FIG. 1, but slide member 23 in this embodiment is limited in its travel by stop 22. The medical device depicted in FIGS. 2 includes a handle with built-in tension 20, a pin vise connector 24, an outer sheath 25, a control rod 26, and a three-prong grasper 27. Pin vise connector 24 connects outer sheath 25 fixedly to handle 20. Handle 20 includes a top portion 20a, a forward or distal portion 20b, an intermediate portion 20c, a gripping and flexible portion 20d, and a rear portion or proximal portion 20e. Rear portion 20e includes terminal portion 20f, which forms an interface with slide member 23.

When a user squeezes handle 20, especially flexible portion 20d, rear portion 20e and interface 20f move distally, causing slide member 23 to also move distally, in the direction of arrow B. Slide member 23, however, does not have complete freedom of movement, and can only move as far in the direction of arrow B as the stop 22 will allow. When the user squeezes handle 20, interface 20f pushes slide member 23 distally, until proximal portion 21 of slide member 23 contacts stop 22, and cannot traverse further distally. The position of stop 22 may be set by having one or more locations tapped in intermediate portion 20c. The throw or distance traversed by slide member 23, and thus control rod 26, is set by selecting the appropriate location in intermediate portion 20c into which stop 22 is threaded or otherwise secured. The handle of FIG. 2 has the same self-tensioning property discussed above with respect to handle 10 in FIG. 1.

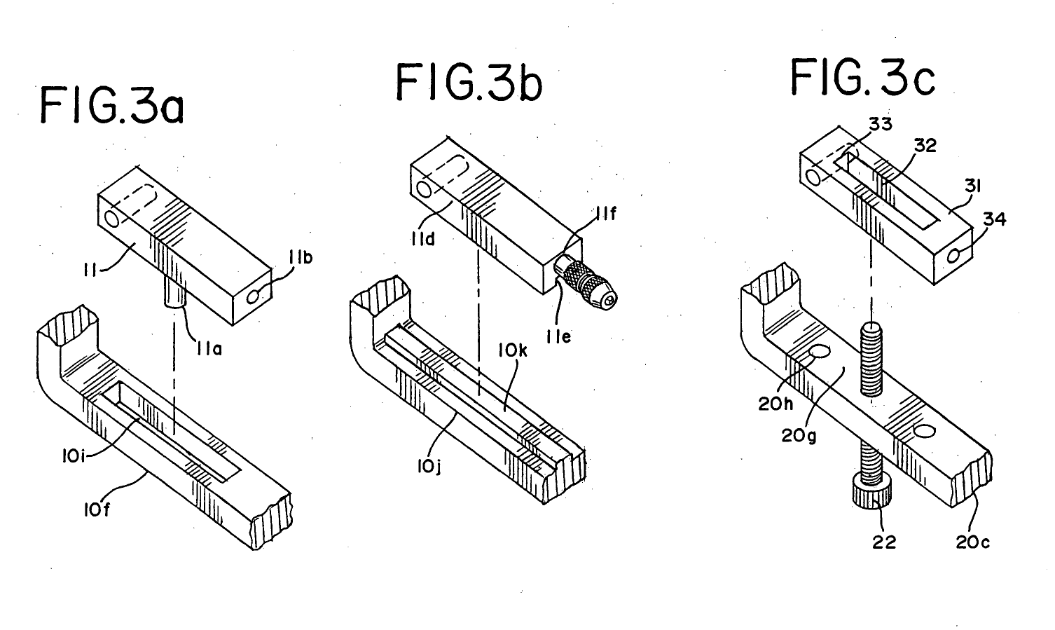

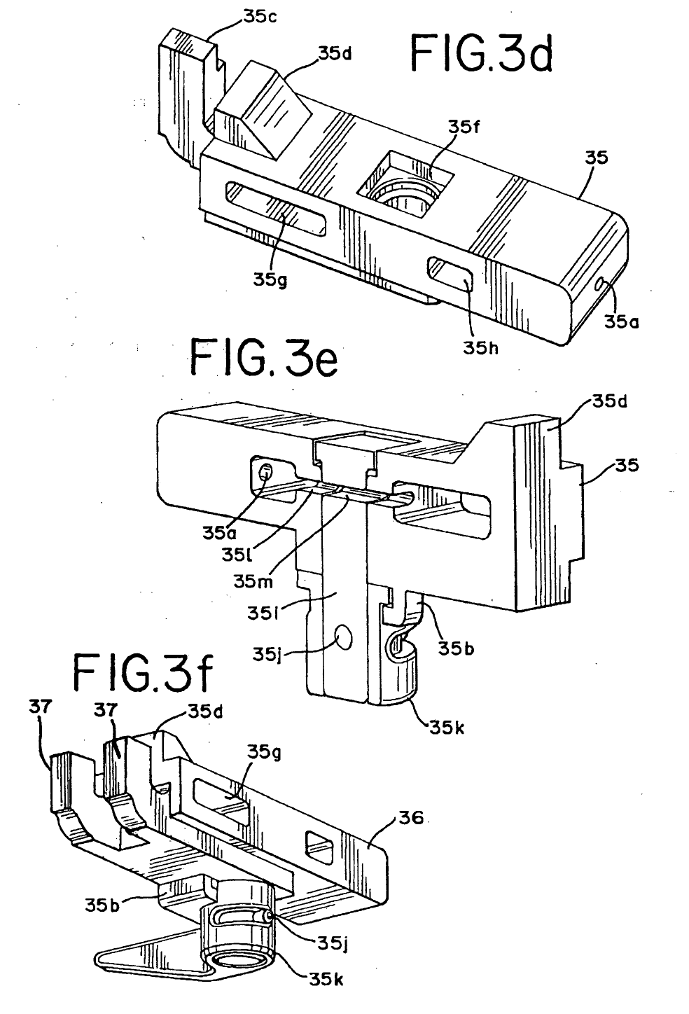

As discussed above, the slide member in the embodiments of FIGS. 1-2 may take on many forms. FIGS. 3a-3f depict several slide member embodiments. FIG. 3a depicts slide member 11. As mentioned above, the performance of the endoscopic device of FIG. 1 is determined by the length of slide member 11f. The slide member preferably also has a threaded aperture 11b or other feature for securing the control rod described above with respect to FIG. 1. The most proximal portion 11c is meant to react against cylindrical member 10h in advancing the slide distally. Slide member 11 bottom area 11a slides between rails or ledges 11i in middle portion 11f of the handle.

Rather than being suspended on rails, the intermediate portion 10j may instead be molded with a ramp or tongue 10k as shown in FIG. 3b. Slide member 11d may then be molded with a groove 11e to match tongue 10k. Slide member 11d slides back and forth in groove 11e by means of tongue 10k. Instead of threading a control rod into slide member 11d, it may be secured with a pin vise 11f. The proximal portions 11c at the rear interact with interface 10h for advancing or retracting the control rod.

FIG. 3c depicts another embodiment of a slide member 31. Slide member 31 includes a slot 32 and a proximal end 33, as well as a distal interface 34 for a control rod. The distal interface may be suitable for an adhesive joint or for any other interface that joins the control rod to the slide member 31. Handle intermediate portion 20c, interfacing with slide member 31, includes a center portion 20g with a series of tapped holes 20h. A bolt 22 is threaded into only one of the tapped holes. When the handle is squeezed, slide member 31 can traverse distally only until proximal end 33 stops when it meets bolt 22. Thus, this embodiment may also be used to control an endoscopic device.

FIGS. 3d-3f depict embodiments of slides useful in the handles of FIGS. 1-2. Slide 35 includes an aperture 35a for a control rod, proximal interfaces 35c, 35d to react cylindrical interface 10h from rear flex portion 10g, and also a proximal aperture 35g and a distal aperture 35h. As shown in partial cross-section in FIG. 3e, vertical opening 35f is used to insert a pin 35i, which is secured to slide 35 with a dowel pin 35j. Pin 35i includes a central shelf 35m which matches shelf 35l, for placement of a cannula or control rod. Pin 35i is moved up or down by cam 35k, which is held in place on the slide by flange 35b. As cam 35k is rotated about 90°, the pin rotates up or down about 0.010 to 0.020 inches. A cannula or control rod in the shelf is secured or released by moving the cam forward or backward. This locking feature yields better control of the cannula and the endoscopic device at the distal end of the cannula. In one modification shown in FIG. 3f, slide 36 may have a proximal interface 37 divided into two portions, the better to control torqueing when the handle is actuated.

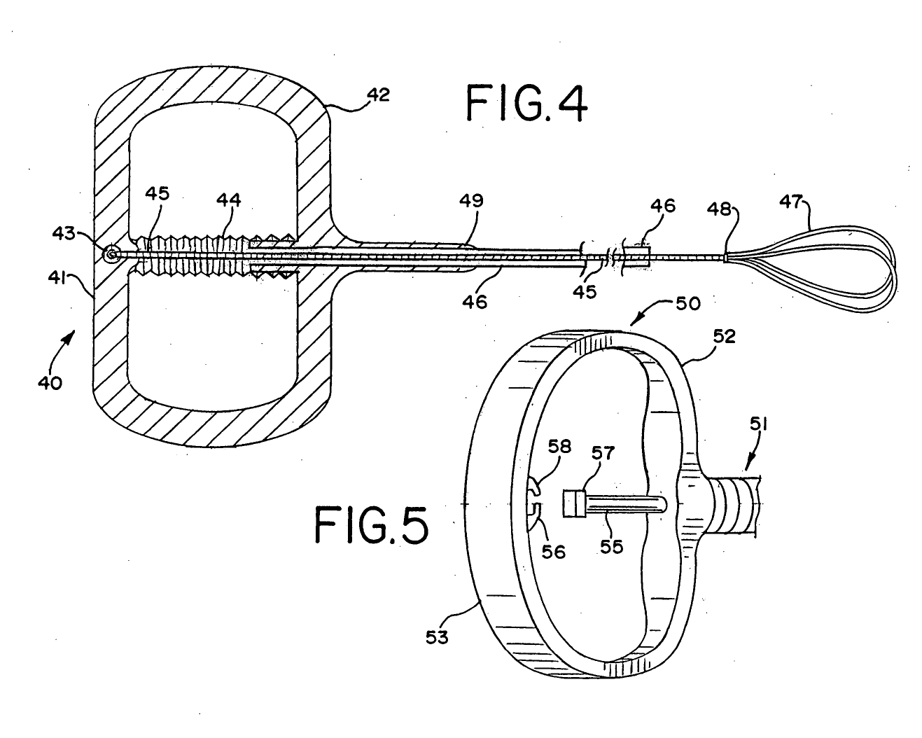

Another embodiment of a self-tensioning handle is shown in FIG. 4, which depicts an endoscopic instrument with a handle 40, an outer sheath 46, a control rod 45, and a retrieval basket 47 made from wires 48, which may be made from a superelastic material or may be made from stainless steel. This self-tensioning handle is preferably molded as a single, continuous, closed plastic molding in the general shape of an oval or rounded rectangle as shown. The rounded rectangle embodiment may be considered to have top, bottom, proximal and distal portions.

The handle 40 includes a proximal portion 41, a distal portion 42, a button screw 43 for securing the control rod, and a protective sleeve 44. The protective sleeve 44 is a thin, flexible sleeve that simply covers and protects the proximal end of control rod 45 that is between distal and proximal portions 41, 42. Outer sheath 46 is secured to the distal end of handle 40 with a pin vise 49. The pin vise fixes the location of sheath 46 with respect to handle 40 and control rod 45. Other devices may be used to attach the control rod and the sheath of an endoscopic instrument.

Squeeze handle 40 operates in a manner similar to that of the embodiment of FIG. 1. A user squeezes the handle, flexing the handle and causing either sheath 46 to retract with respect to control rod 45, or causing control rod 45 to extend relative to sheath 45. In practice, handle 40 may be designed for either mode of operation, by making either proximal portion 41 or distal portion 42 more flexible. If proximal portion 41 is more flexible, when a user squeezes handle 40, proximal portion 41 will deform and move distally, to the right in FIG. 4, causing control rod 45 to extend distally, and deploying basket 47. If distal portion 42 is more flexible, then a user's squeeze will cause distal portion 42 to move to the left in FIG. 4, in a proximal direction, causing sheath 46 to also move proximally, exposing basket 47 and wires 48.

The desired flexibility may be accomplished by using different materials in the distal and proximal portions, or by designing the distal and proximal portions with differing dimensions, so that one portion is more flexible, and more deformable, than the other. For instance, if it is desired to make proximal portion 41 more flexible, a plastic with a lower modulus of elasticity may be used to make proximal portion 41, while a stiffer plastic, having a higher modulus of elasticity, may be used for distal portion 42. Alternatively, distal portion 42 may be made thicker or broader in one or both cross-sectional dimensions, while proximal portion 41 may be thinner or less broad than distal portion 42.

Sheath 46 may be secured to handle 40 with a pin vise connection 49, as shown, or with any other suitable connection that holds sheath 46 fixedly in place. Control rod 45 may be secured to handle 40 with a button screw 43 as shown, or control rod 45 may be secured in any other desired manner. For instance, control rod 45 may have a T-shape or other retaining shape at its proximal end for easy insertion and removal into proximal portion 41 of the handle. The handle is provided with a self-tensioning property by molding the handle and assembling the endoscopic device such that when proximal portion 41 is squeezed to expose control rod 456 and basket 47, the handle pulls back naturally, providing tension on the control rod and tending to pull the control rod in a proximal direction.

Another embodiment of a self-tensioning handle is depicted in FIG. 5. Handle 50 is generally oval in shape, with a vertical axis somewhat longer than the horizontal axis, and having to and bottom portions as shown connecting the distal and proximal portions of the handle. In one embodiment, distal portion 52 may be somewhat stiffer than more flexible proximal portion 53. Distal portion 52 may also include a pin-vise type tip 51 or other device for fixedly attaching a sheath. Handle 50 may also include an interface 56 with a shaped opening 58. Opening 58 will preferably be shaped for easy introduction of a control rod 55 and its correspondingly-shaped proximal end 57. As mentioned above, the handle is flexible and is made self-tensioning by the above-mentioned assembly technique. When a user squeezes the handle, and extends the control rod or retracts the sheath, the flexibility of the handle provides a tension that automatically retracts the control rod or extends the sheath, thus providing at least momentary relief for the operator of the endoscopic device.

To use handle 50, a user inserts a control rod 55 through distal portion 52 and attaches a sheath (not shown) fixedly to distal tip 51. The user then inserts proximal end 57 of control rod 55 to interface 56 and firmly presses proximal end 55 into the interface. When the user wishes to extend control rod 55 and any end-effector at the distal end of the control rod, the user squeezes the handle. Flexible proximal portion 53 deforms by bending inwardly, to the right in FIG. 5, and control rod 55 is also pushed to the right, extending the end-effector or basket at the distal end of the control rod.

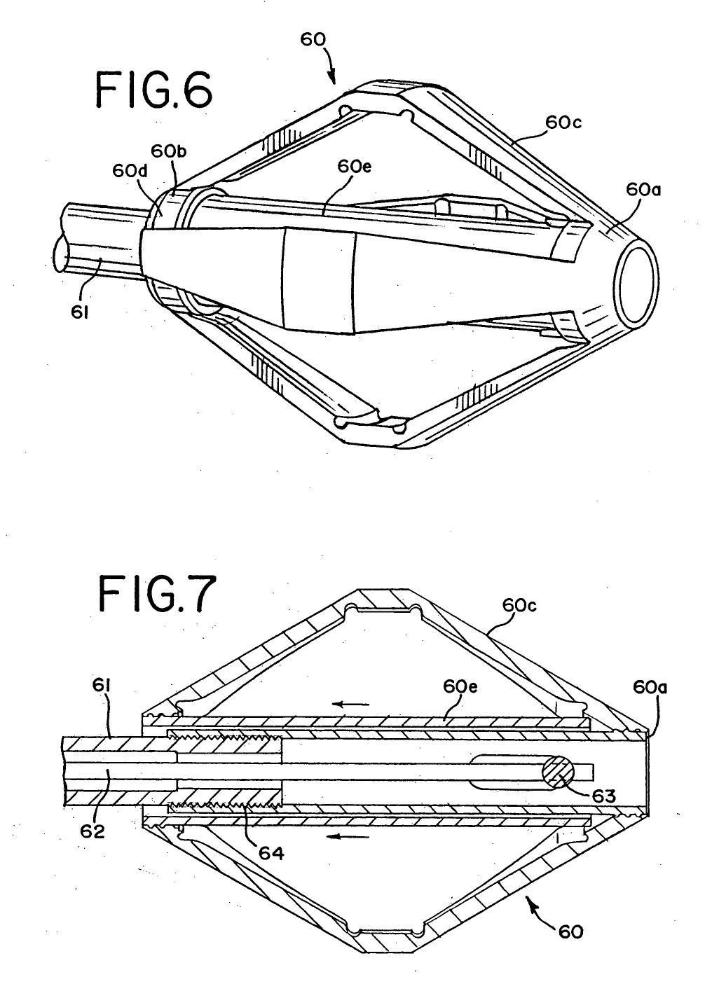

Another embodiment of a molded squeezable handle is depicted in FIGS. 6-7. Handle 60 comprises a proximal end 60a, proximal interface 60d, distal end 60b, a distal interface 60e, and a plurality of bendable sections 60c. The plurality of bendable sections 60c may be considered to be a squeezable actuation portion comprising at least two webs 60c between proximal and distal ends 60a, 60b. There may also preferably be at least one spacer 60f for use between distal interface 60e and a sheath 61 used in conjunction with handle 60. The arrangement of the parts is best seen in FIG. 7. Any one of the web sections 60c may be considered to be a top portion, with an opposite web section being a bottom portion.

The outer portion of the handle, the portion that is touched by the hands of a user, includes ends 60a, 60b and the bendable sections 60c. These may be molded as a single unit, preferably from a plastic material that is flexible or bendable, such as polyethylene or polypropylene. A user squeezes the handle, causing either a sheath to retract or a control rod or interface to extend. This flexible handle may be used in either manner. In the embodiment shown in FIGS. 6-7, control rod 62 is connected to distal interface 60e by pin 63, while sheath 61 is adhered to proximal interface 60d by a glue joint 64.

A user deploys the end-effector (not shown) at the distal end of control rod 62 by squeezing bendable sections 60c. When these sections flex and bend, distal interface 60e moves left, in the direction of arrow A in FIG. 7. Sheath 61 is fixed by glue joint 64 and proximal interface 60d, while distal interface 60e moves with pin 63 and control rod 62. Those skilled in the art will recognize that the handle may alternatively be configured so that squeezing moves one of the sheath and the control rod to the right, i.e., with the control rod is fixedly mounted to the handle, while the sheath is moved to the right or retracted by squeezing the handle. In this manner, the handle may be configured to either extend the control rod and an end-effector mounted at the distal end of the control rod, while the sheath remains fixed. Alternatively, the sheath may be retracted, thereby deploying the end-effector. The present self-tensioning invention is meant to include both embodiments.

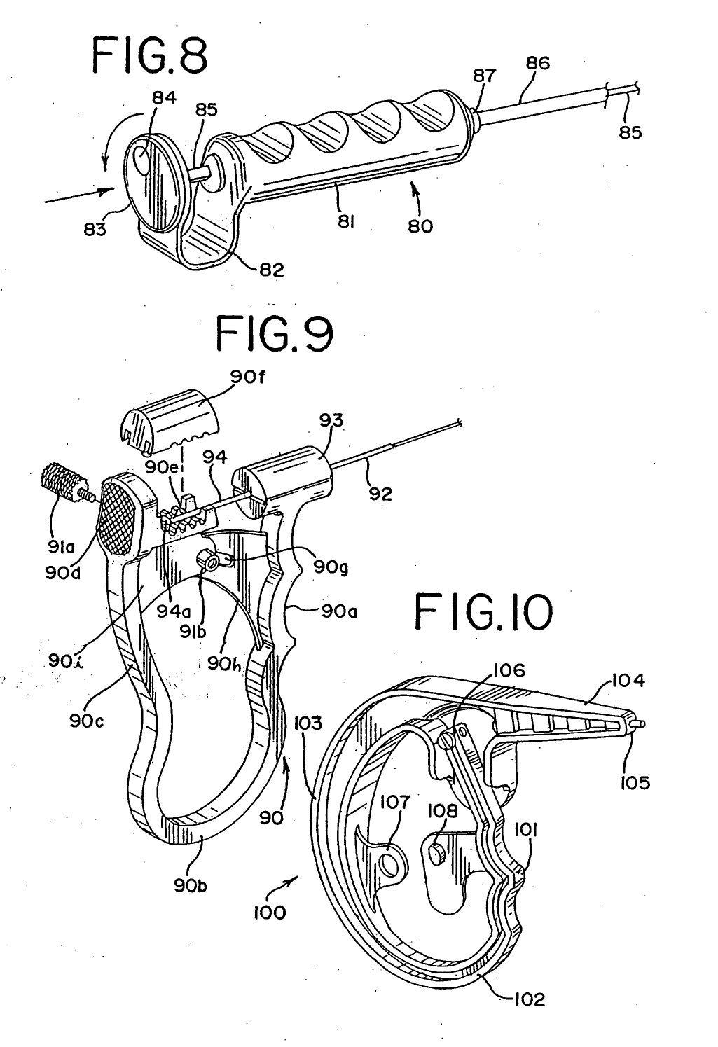

Another embodiment of a squeeze handle is depicted in FIG. 8. This squeeze handle 80 comprises a gripping portion 81, a bendable portion 82, and a separate, rotatable control portion 83. Rotatable portion 83 may include a thumb-rest 84 for further control by a user. Gripping portion 81 may also include finger-mounting portions 81a. In this embodiment, a sheath 86 is fixedly mounted to the distal end 87 of the handle, while a control rod 85 is mounted to control portion 83.

Handle 80, also a self-tensioning handle, functions in much the same manner as handle 40, depicted in FIG. 4. A user advances control rod 85 by squeezing control portion 83, thus flexing bendable portion 82. In one embodiment, the proximal end of control rod 85 may be T-shaped, as shown in FIG. 5, so that when a user rotates control portion 83 using thumb-rest 84, the control rod is also rotated. This enables the surgeon to maneuver an end-effector (not shown) at the end of control rod 85. By maneuvering the end-effector, such as a basket or a grasper, the surgeon may avoid obstacles or capture objects within a patient, such as kidneystone fragments or other undesirable objects.

In other embodiments, the squeezable, self-tensioning handle may add more components for better control by a user, as shown in FIGS. 9-10. In FIG. 9, the control portion may be molded or machined with specific increments for holding a control rod, so that squeezing the handle will advance the control rod, and hence the end-effector, a known distance. The handle will naturally tend to pull back, thus imparting a self-tensioning property to handle 90. Squeezable handle 90 includes a proximal portion 90a with positions for the fingers of a user, a bendable portion 90b, and a distal or palm portion 90c. Distal portion 90c may also include a thumb rest 90d and an adjustable control portion 90e.

As shown, control portion 90e may include one or more specific rests for a proximal portion 94a of a control rod 94. As discussed above, proximal portion 94a is preferably T-shaped, or shaped in some other manner for ease of placement in a manner that allows a user to displace and maneuver control rod 94. To assemble a retrieval basket or other endoscopic instrument to the handle, control rod 94 is placed into one the of resting places in control portion 90e and cap 90f is placed over the control portion. Sheath 92 is fixedly mounted to sheath mount 95 and cap 93 may also be placed over sheath mount 95. Caps 90f and 93 may snap-fit onto their respective portions, or they may mount in any suitable manner.

A surgeon or other user of handle 90 may wish to fix the position of the retrieval basket, grasper, or other end-effector used with the handle. Handle 90 has webs 90h and 90i, affixed to the distal and proximal portions respectively. Web 90h may have a arced slot 90g while proximal web 90i has an aperture (not shown) and a threaded retainer or nut 91b. A user may squeeze the handle until the end-effector at the distal end of control rod 94 is in a desired position, and may then fix the position of control rod 94 and the end-effector by threading a retaining bolt 91a into nut 91d, locking the position of the control rod. In this manner, the surgeon may rest momentarily, or may free his hands for another task related to the patient at hand. Alternatively, the bolt and nut may be assembled onto proximal web 90i and used as a “stop,” to set a maximum traverse or “throw” of control rod 94, thus limiting the travel of the retrieval basket or other device at the distal end of control rod 94.

The squeeze handle depicted in FIG. 10 also includes more components so that a surgeon using the handle has more freedom in maneuvering an endoscopic device. Handle 100 includes a proximal or finger portion 101, a bendable portion 102, and a distal or palm portion 103. Control portion 104 includes a mounting portion 105 for fixedly mounting one portion of an end-effector (not shown). Proximal portion 101 includes a mount 106 for mounting another portion of an end-effector (not shown) and also includes a proximal locking portion 108. Proximal locking portion 108 may be used with distal locking portion 107 to fix the position of the handle and thus the end-effector used with the handle. As mentioned above, this allows the surgeon freedom to rest or to perform another task while temporarily resting the squeezable handle and the endoscopic device used with the handle. The handle may be considered to have a top portion comprising control portion 104 and mounting portion 105, and a bottom portion that includes bendable portion 102.

Mounting portion 105 may mount a control rod or a sheath, and mount 106 may mount the other of a control rod or a sheath. The control rod is advanced, or the sheath is retracted, by squeezing handle 100. A self-tensioning property is imparted by proper assembling of an endoscopic device to the handle. When handle 100 is assembled, pivot pin 109 on the proximal portion fits into slot 110 of control portion 104. In this embodiment, the position of the sheath or the control rod is adjusted by squeezing handle 100. As mentioned above, the position of the handle may be fixed by locking portions 107, 108 to each other.

Accordingly, it is the intention of the applicants to protect all variations and modifications within the valid scope of the present invention. It is intended that the invention be defined by the following claims, including all equivalents. Since the foregoing detailed description has described only a few of the many alternative forms this invention can take, it is intended that only the following claims, including all equivalents, be regarded as a definition of this invention.

1. A self-tensioning handle for an endoscopic device, the handle comprising:
an upper portion;
an intermediate portion mounted to the upper portion; and
an actuating portion operably connected to the adjustable portion for operating the endoscopic device, wherein the handle is configured to fit in a palm of a user's hand and the upper, intermediate, and actuating portions are molded together.

2. A handle according to claim 1, further comprising a sliding portion attached to the intermediate portion.

3. A handle according to claim 1, wherein the intermediate portion further comprises a slot, and further comprising a sliding portion with a retainer sliding in the slot, wherein the sliding portion controls a throw of the handle.

4. A handle according to claim 1, further comprising a sliding portion attached to the intermediate portion, the sliding portion configured for attachment of a control rod for the endoscopic device.

5. A handle according to claim 1, further comprising a sliding portion and a pin in an aperture of the sliding portion, and a cam, the sliding portion riding in the intermediate portion, the pin moving up and down in response to the cam for gripping and releasing a control rod.

6. A handle according to claim 1, further comprising a sliding portion attached to the intermediate portion, the sliding portion comprising a divided rear portion.

7. An endoscopic device with a handle according to claim 1, further comprising an end-effector mounted to the handle.

8. A self-tensioning handle for an endoscopic device, the handle comprising:
an upper portion;
an intermediate portion mounted to the upper portion;
an adjustable portion mounted to the intermediate portion; and
an actuating portion operably connected to the adjustable portion for operating the endoscopic device, wherein the handle is configured to fit in a palm of a user's hand.

9. A handle according to claim 8, wherein the upper portion, intermediate portion, and actuating portion are molded from a plastic material as a unit.

10. A handle according to claim 8, wherein the intermediate portion further comprises a device for fixedly mounting a first portion of the endoscopic device and the adjustable portion comprises a device for mounting a second portion of the endoscopic device.

11. A handle according to claim 8, wherein the device for fixedly mounting the first portion is a button screw and the device for fixedly mounting the second portion is a pin vise.

12. A self-tensioning handle for controlling an endoscopic device, the handle comprising:
a proximal portion for mounting a first portion of the endoscopic device;
a distal portion for mounting a second portion of the endoscopic device; and
a squeezable actuation portion comprising at least two webs attached to the proximal and distal portions.

13. A handle according to claim 12, wherein the squeezable actuation portion comprises at least one of a distal and a proximal portion.

14. A handle according to claim 12, wherein at least one of the first and second portions are attached to the handle with an adhesive.

15. A handle according to claim 12, wherein at least one of the first and second portions are attached to the handle with a fastener.

16. A handle according to claim 12, further comprising an end-effector for mounting to the proximal and distal portions.

17. An endoscopic device comprising a handle according to claim 12, and further comprising an end effector attached to the distal and proximal portions.

18. A self-tensioning handle for an endoscopic device, the handle comprising:
a proximal portion for mounting a first portion of the endoscopic device;
a distal portion for mounting a second portion of the endoscopic device;
a bendable portion connecting the proximal and distal portions; and
at least one control device for controlling movement of the endoscopic device, wherein the proximal, flexible and distal portions are molded together.

19. A handle according to claim 18, wherein the proximal portion of the handle comprises a hand-grip with indentations for fingers of a user.

20. A handle according to claim 18, wherein the at least one control device is selected from the group consisting of a rotatable control portion, an adjustable control portion, and a locking portion.

21. A handle according to claim 18, wherein the proximal and distal portions each comprise a web and further comprising a fastener for fixing a position of one of the proximal and distal portions with respect to the other of the proximal and distal portions for controlling the endoscopic device.

22. A handle according to claim 18, wherein at least one of the proximal and distal portions comprises a control portion for adjustably mounting a portion of the endoscopic device.

23. An endoscopic device with a handle according to claim 18, further comprising an end effector for mounting to the proximal and distal portions.
